(19) United States
(12) Patent Application Publication
(10) Pub. No.: US 2025/0107837 A1
(43) Pub. Date: Apr. 3, 2025

(54) LOW FREQUENCY RF ABLATION SYSTEM

(71) Applicant: Novocure GmbH, Root D4 (CH)

(72) Inventor: Igal Balin, Haifa (IL)

(21) Appl. No.: 18/894,349

(22) Filed: Sep. 24, 2024

Related U.S. Application Data
(60) Provisional application No. 63/586,816, filed on Sep. 29, 2023.

(51) Int. Cl. A61B 18/10; A61B 18/00; A61B 18/08; A61N 1/36

(52) U.S. Cl. CPC A61B 18/10; A61B 18/082; A61B 2018/00577; A61B 2018/00642; A61B 2018/00678; A61B 2018/00714; A61B 2018/00732; A61B 2018/00815; A61N 1/36002

(57) ABSTRACT

Systems, methods of use, and kits are herein described, including a method comprising applying one or more pair of transducer arrays to a skin surface of a patient, placing a probe within the patient such that a probe tip is disposed at a designated location within the patient, and activating an electric field generator to supply an alternating current (AC) electrical signal to the one or more pair of transducer arrays. The designated location is between the one or more pair of transducer arrays. The probe tip is configured to heat when disposed within an electric field. The AC electrical signal has a frequency in a range from 50 kHz to 1 MHz. The or more pair of transducer arrays receiving the AC electrical signal generates an electric field between the one or more pair of transducer arrays such that the designated location is within the electric field.

### REFERENCE TO RELATED APPLICATIONS

The present patent application claims priority to the United States provisional application identified by U.S. Ser. No. 63/586,816, filed on Sep. 29, 2023. The entire content of the provisional patent application is hereby incorporated herein by reference.

### BACKGROUND ART

Tumor Treating Fields (TTFields or TTFs) are low intensity (e.g., 1-3 V/cm) alternating electric fields within the intermediate frequency range (e.g., 50 kHz to 1 MHz, such as 50-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. More specifically, for the OPTUNE® system, one pair of electrodes of the transducer array is located to the left and right (LR) of the tumor, and the other pair of electrodes of the transducer array is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a combination therapy with chemotherapy for malignant pleural mesothelioma (MPM), and may find use in treating tumors in other parts of the body.

### SUMMARY OF THE INVENTION

A method and system are disclosed. In one aspect, the present disclosure relates to a method, comprising: applying one or more pair of transducer arrays to a skin surface of a patient; placing a probe within the patient such that a probe tip of the probe is disposed at a designated location within the patient, the designated location being between the one or more pair of transducer arrays, the probe tip configured to heat when disposed within an electric field; and activating an electric field generator to supply an alternating current (AC) electrical signal having a frequency in a range from 50 kHz to 1 MHz to the one or more pair of transducer arrays, thereby generating the electric field between the one or more pair of transducer arrays such that the designated location is within the electric field for a period of time.

In another aspect, the present disclosure relates to a system, comprising: an electric field generator operable to generate an AC electrical signal having a frequency in a range from 50 kHz to 1 MHz; one or more pair of transducer arrays configured to be electrically connected to the electric field generator and operable to generate an electric field based on the AC electrical signal; and a probe having a probe tip sized and dimensioned for insertion into a patient's body.

In another aspect, the present disclosure relates to a kit, comprising: an electric field generator operable to generate an AC electrical signal having a frequency in a range from 50 kHz to 1 MHz; a pair of transducer arrays configured to be electrically connected to the electric field generator and operable to generate an electric field based on the AC electrical signal; and a probe having a probe tip operable to generate heat when disposed within the electric field.

In another aspect, the present disclosure relates to a non-transitory processor-readable medium storing processor-executable instructions that when executed by a processor cause the processor to: obtain a model of AC electrical conductivity in a portion of a patient's body including a region of interest; determine a plurality of heating potentials with each heating potential corresponding to a probe tip location and a pair of transducer array locations, by using the model of AC electrical conductivity to simulate an indication of the heating potential of the region of interest for a plurality of probe tip locations within the patient's body adjacent to the region of interest and particular pairs of transducer array locations of a plurality of pairs of transducer array locations encompassing the region of interest when an electric field is induced between the particular pairs of transducer array locations; select one or more recommended pair of transducer array locations and one or more corresponding recommended probe tip location based at least in part on the model of AC electrical conductivity and one or more corresponding heating potential; and output the one or more recommended pair of transducer array locations and the one or more corresponding recommended probe tip location.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a diagrammatic view of exemplary embodiments of electrodes as applied to living tissue in accordance with the present disclosure;

FIG. 2A is a diagrammatic view of an exemplary embodiment of an electronic device configured to generate a TTField constructed in accordance with the present disclosure;

FIG. 2B is a diagrammatic view of another exemplary embodiment of the electronic device shown in FIG. 2A;

FIG. 3 is a diagrammatic view of exemplary embodiments of transducer array locations and probe tip locations of a patient's body in accordance with the present disclosure; and

FIG. 4 is a diagrammatic view of an exemplary embodiment of a method of ablating a target tissue in accordance with the present disclosure;

FIG. 5 is a diagrammatic view of an exemplary embodiment of a method of providing recommendations for transducer array placement and ablation probe placement in accordance with the present disclosure;

FIG. 6A is a top view of an exemplary embodiment of an experimental arrangement for performing validation experiments in accordance with the present disclosure;

FIG. 6B is a front perspective view of the experimental arrangement shown in FIG. 6A; and

FIG. 7 is a graph of experimental test results determined using the experimental arrangement shown in FIGS. 6A and 6B.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context.

Unless otherwise required by context, singular terms shall include pluralities and plural terms shall include the singular.

All of the compositions, assemblies, systems, kits, and/or methods disclosed herein can be made and executed without undue experimentation in light of the present disclosure. Where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

The use of the term “a” or “an” when used in conjunction with the term “comprising” in the claims and/or the specification may mean “one,” but is also consistent with the meaning of “one or more,” “at least one,” and “one or more than one.” The term “plurality” refers to “two or more.”

In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task. The processor may communicate with a non-transitory computer-readable medium storing computer-executable instructions that when executed by the processor causes the processor to perform a specified function. Exemplary non-transitory computer-readable mediums may include a non-volatile memory, a random-access memory (RAM), a read only memory (ROM), a CD-ROM, a hard drive, a solid-state drive, a flash drive, a memory card, a DVD-ROM, a Blu-Ray Disk, a laser disk, a magnetic disk, an optical drive, combinations thereof, and/or the like.

As used herein, the term TTField (TTFields, or TTF(s)) refers to low intensity (e.g., 1-4 V/cm) alternating electric fields of medium frequencies (about 50 kHz-1 MHz, and more preferably from about 50 kHz-500 kHz) that when applied to a conductive medium, such as a human body, via electrodes may be used, for example, to treat tumors as described in U.S. Pat. No. 7,016,725, 7,089,054, 7,333,852, 7,565,205, 7,805,201, and 8,244,345 by Palti (each of which is incorporated herein by reference) and in a publication by Kirson (see Eilon D. Kirson, et al., Disruption of Cancer Cell Replication by Alternating Electric Fields, Cancer Res. 2004 64:3288-3295). TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients.

The term “transducer array”, as used herein, means a conductive transducer array or a non-conductive transducer array. Exemplary transducer arrays may include, for example, pads disclosed in any one of U.S. Patent Publication No. 2021/0346693 entitled “CONDUCTIVE PAD GENERATING TUMOR TREATING FIELD AND METHODS OF PRODUCTION AND USE THEREOF” and U.S. Patent Application No. 63/128,265 entitled “OPTIMIZATION OF COMPOSITE ELECTRODE”, all of which are hereby expressly incorporated by reference herein in their entirety.

As used herein, all numerical values or ranges include fractions of the values and integers within such ranges and fractions of the integers within such ranges unless the context clearly indicates otherwise. The numerical ranges specified herein includes the endpoints, and all values, sub-ranges of values within the range, and fractions of the values and integers within said range. Thus, any two values within the range of 1 mm to 10 m, for example, can be used to set a lower and an upper boundary of a range in accordance with the embodiments of the present disclosure.

Referring now to the drawings and in particular to FIG. 1, shown therein is an exemplary embodiment of a dividing cell 10, under the influence of external TTFields, generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The microtubules 22 positive charges are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions. In one embodiment, the cells go through electroporation, that is, DNA or chromosomes are introduced into the cells using a pulse of electricity to briefly open pores in the cell membranes.

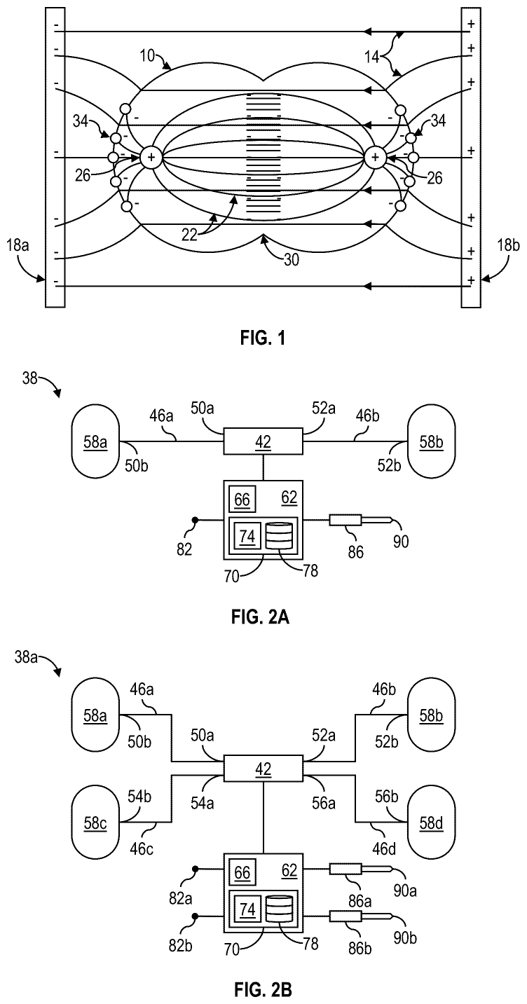

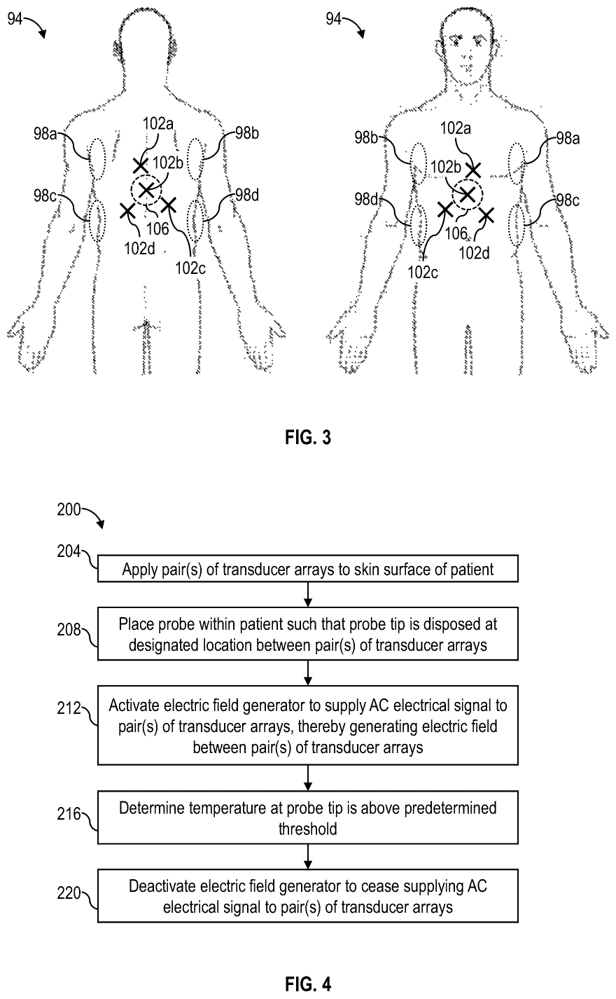

Referring now to FIG. 2A, shown therein is an exemplary embodiment of a system 38 for applying TTFields to a patient 94 (shown in FIG. 3) constructed in accordance with the present disclosure. The system 38 generally comprises an electric field generator 42 and a plurality of conductive leads, shown in FIG. 1 as a first conductive lead 46a and a second conductive lead 46b (collectively, the “conductive leads 46”). While the system 38 is shown in FIG. 1 as comprising two of the conductive leads 46, in other implementations, the system 38 comprises more or less than two of the conductive leads 46.

The electric field generator 42 is operable to supply power and generate an electrical signal (i.e., a TTField signal), which may be an alternating current (AC) electrical signal or a pulsed electrical signal having a frequency in a range from 50 kHz to 1 MHz (or, preferably, from 100 kHz to 500 kHz). A voltage of the TTField signal may be such that an electric field (i.e., a TTField) in tissue within a treatment area has an intensity in a range from 0.1 V/cm to 10 V/cm.

The first conductive lead 46a has a first end 50a and a second end 50b, and the second conductive lead 46b has a first end 52a and a second end 52b. The conductive leads 46 are isolated conductors with a flexible metal shield and are preferably grounded, thereby preventing spread of any electric field generated by the conductive leads 46.

Each of the conductive leads 46 are electrically connected to the electric field generator 42. Accordingly, the first end 50a of the first conductive lead 46a and the first end 52a of the second conductive lead 46b are electrically connected to the electric field generator 42. The conductive leads 46 are also electrically connected to a plurality of transducer arrays, shown in FIG. 1 as a first transducer array 58a and a second transducer array 58b (collectively, the “transducer arrays 58”). Accordingly, the second end 50b of the first conductive lead 46a and the second end 52b of the second conductive lead 46b are electrically connected to the first transducer array 58a and the second transducer array 58b, respectively.

Each of the transducer arrays 58 is supplied with the TTField signals. The transducer arrays 58, being supplied with the TTField signals, cause an electrical current to flow between the transducer arrays 58, thereby causing the TTField to be generated between the transducer arrays 58. The transducer arrays 58, when applied to a treatment area on a patient 94 (shown in FIG. 2), cause the TTField to be generated in a region of interest 106 (shown in phantom in FIG. 2) within the patient 94. The target region may include one or more tumor, and the generation of the TTField may selectively destroy and/or inhibit growth of the at least one tumor. While the system 38 is shown in FIG. 1 as comprising two of the transducer arrays 58, in other implementations, the system 38 comprises more or less than two of the transducer arrays 58.

To optimize a distribution of the TTField, the transducer arrays 58 may be configured differently based on a particular application in which the transducer arrays 58 are used. Further, the transducer arrays 58 may have specific shapes and positions so as to generate the TTField having a desired configuration, direction, and/or intensity at the treatment area and only at the treatment area so as to focus the treatment in the target region. A user may apply the transducer arrays 58 to the treatment area on the patient 94 (i.e., applied to a skins surface of the patient 94) to cause the TTField to be generated within the target region. The TTField may be of a widely distributed type or a local type (e.g., to treat skin tumors or lesions close to the skin surface).

The user may be a medical professional, such as a doctor, nurse, caregiver, therapist, or other person acting under the instruction of a doctor, nurse, caregiver, or therapist. In some embodiments, the user may be the patient 94 (i.e., the patient 94 and/or a helper may apply the transducer arrays 58 to the treatment area).

In some embodiments, the system 38 further comprises a controller 62. The controller 62 generally comprises circuitry operable to control an output of the electric field generator 42, for example, to set the output between a minimal value that causes ablation of the treatment area and a maximal value that does not cause excessive heating of the treatment area. The controller 62 may issue a warning or the like when a temperature of the treatment area (as sensed by one or more temperature sensor 82 (hereinafter the “temperature sensor 82”), discussed in more detail below) exceeds a preset limit. The temperature sensor 82 may be mechanically connected to and/or otherwise associated with one or more ablation probe 86 (hereinafter the “ablation probe 86”) so as to sense the temperature of the treatment area adjacent to a probe tip 90 of the ablation probe 86 as described below in more detail. The ablation probe 86 may be in the form of a rod or a needle constructed from a conductive and biocompatible material, such as stainless steel or titanium.

The temperature sensor 82 is generally configured to measure a temperature. In some embodiments, the temperature sensor 82 is a thermistor (i.e., a variable resistor having a resistance that varies based on temperature). In such embodiments, the controller 62 may receive a resistance reading from the temperature sensor 82 indicative of the resistance of the temperature sensor 82.

The probe tip 90 of the ablation probe 86 may be sized and dimensioned for insertion into the body of the patient 94. In use, a user may insert the probe tip 90 of the ablation probe 86 into the body of the patient 94 such that the probe tip 90 is disposed at a designated location. Without being bound by theory, it is believed that the probe tip 90 being disposed in an electric field may cause an intensity of the electric field to increase immediately adjacent to the probe tip 90. The increased intensity of the electric field immediately adjacent to the probe tip 90 may cause thermal energy to be delivered to a target tissue adjacent to the probe tip 90, thereby treating, removing, or destroying the target tissue.

In some embodiments, the circuitry of the controller 62 includes one or more processor 66 (hereinafter the “processor 66”) and one or more non-transitory processor-readable medium 70 (hereinafter the “memory 70”). The memory 70 may store processor-executable instructions 74 and/or a datastore 78. The processor-executable instructions 74 when executed by the processor 66 may cause the processor 66 to perform one or more action described herein. The processor 66 may be in communication with the temperature sensor 82, the ablation probe 86, and/or other circuitry (e.g., an analog-to-digital converter, a digital-to-analog converter, a multimeter, an ohmmeter, a voltmeter, and/or an ammeter), for example. In some embodiments, the processor-executable instructions 74 when executed by the processor 66 may cause the processor 66 to perform one or more step of the methods described herein.

In some embodiments, the controller 62 may turn off or decrease power of the TTField signals generated by the electric field generator 42 if a temperature sensed by the temperature sensor 82 meets or exceeds a comfortability threshold. In one embodiment, the comfortability threshold is the temperature at which a patient 94 would be made uncomfortable while using the first transducer array 58a, the second transducer array 58b, and the ablation probe 86. For example, the comfortability threshold may be a temperature at or about 40 degrees Celsius. In one embodiment, the comfortability threshold is a temperature of between about 39 degrees Celsius and 42 degrees Celsius, or a specific selected temperature between about 39 degrees Celsius and 42 degrees Celsius.

In some embodiments, the controller 62 may turn off or decrease power of the TTField signals generated by the electric field generator 42 after a sufficient period of time has elapsed for the probe tip 90 to heat to a therapeutic temperature (i.e., a temperature adequate for ablation).

Referring now to FIG. 2B, shown therein is another exemplary embodiment of the system 38a shown in FIG. 2A. In some embodiments, the conductive leads 46 include the first conductive lead 46a, the second conductive lead 46b, a third conductive lead 46c, and a fourth conductive lead 46d. In such embodiments, the third conductive lead 46c has a first end 54a electrically connected to the electric field generator 42 and a second end 54b electrically connected to a third transducer array 58c, and the fourth conductive lead 46d has a first end 56a electrically connected to the electric field generator 42 and a second end 56b electrically connected to a fourth transducer array 58d.

In some embodiments with the third transducer array 58c and the fourth transducer array 58d, the processor 66 may be communication with a first temperature sensor 82a and a second temperature sensor 82b. The first temperature sensor 82a may mechanically connected to and/or otherwise associated with a first ablation probe 86a having a first probe tip 90a, and the second temperature sensor 82b may mechanically connected to and/or otherwise associated with a second ablation probe 86b having a second probe tip 90b.

Referring now to FIG. 3, shown therein are a plurality of potential transducer array locations, such as a first potential transducer array location 98a, a second potential transducer array location 98b, a third potential transducer array location 98c, and a potential fourth transducer array location 98d (collectively, the “potential transducer array locations 98”), and a plurality of potential probe tip locations, such as a first potential probe tip location 102a, a second potential probe tip location 102b, a third potential probe tip location 102c, and a fourth potential probe tip location 102d (collectively, the “potential probe tip locations 102”).

The potential transducer array locations 98 and the potential probe tip locations 102 may be determined based on a region of interest 106. That is, one or more of the potential probe tip locations 102 may be determined to be adjacent to the region of interest 106 and one or more of the potential transducer array locations 98 may be determined to be encompassing the region of interest 106. The region of interest 106 may include a target tissue selected for ablation using the system 38. While the potential transducer array locations 98 and the potential probe tip locations 102 are shown in FIG. 3 as being on or in the torso of the patient 94, it should be understood that the potential transducer array locations 98 and the potential probe tip locations 102 may be on or in any part of the body of the patient 94.

As described herein, a pair of potential transducer array locations 98 may include any combination of two of the potential transducer array locations 98. The region of interest 106 may be between the combination of two potential transducer locations 98. For example, a pair of potential transducer array locations 98 may include any two of the first potential transducer array location 98a, the second potential transducer array location 98b, the third potential transducer array location 98c, and the potential fourth transducer array location 98d.

Referring now to FIG. 4, shown therein is an exemplary embodiment of a method 200 of ablating a target tissue in accordance with the present disclosure. As shown in FIG. 4, the method 200 generally comprises the steps of: applying one or more pair of transducer arrays 58 to a first position (i.e., any two of the potential transducer array locations 98) on a skin surface of a patient 94 (step 204); placing an ablation within the patient 94 such that a probe tip 90 of the ablation probe 86 is disposed at a designated location (i.e., one of the potential probe tip locations 102) within the patient 94 (step 208); and activating an electric field generator to supply an AC electrical signal having a frequency in a range from 50 kHz to 1 MHz to the one or more pair of transducer arrays, thereby generating the electric field between the one or more pair of transducer arrays 58 such that the designated location is within the electric field for a period of time (step 212).

The period of time may be at least a sufficient period of time for the probe tip 90 of the ablation probe 86 to heat to a therapeutic temperature (i.e., a temperature adequate for ablation). In some embodiments, the method 200 further comprises, subsequent to the period of time elapsing, deactivating the electric field generator 42 to cease supplying the AC electrical signal to the one or more pair of transducer arrays 58.

In some embodiments, the method 200 may further comprise, subsequent to deactivating the electric field generator 42, moving the one or more pair of transducer arrays 58 to a second position (i.e., another two of the potential transducer array locations 98) on the skin surface of the patient 94.

In some embodiments where the system 38a includes a first ablation probe 86a and a second ablation probe 86b, the method 200 may further comprise placing the second ablation probe 86b within the patient 94 such that a second probe tip 90b of the second ablation probe 86b is disposed at a second designated location (i.e., another one of the potential probe tip locations 102) within the patient 94.

In some embodiments, the method 200 may further comprise activating the electric field generator 42 to supply the AC electrical signal having the frequency in the range from 50 kHz to 1 MHz to the one or more pair of transducer arrays 58 in the second position, thereby generating the electric field between the one or more pair of transducer arrays 58 in the second position such that the second designated location is within the electric field for a second period of time.

In some embodiments where the system 38a includes a first pair of transducer arrays 58 (e.g., the first transducer array 58a and the second transducer array 58b) and a second pair of transducer arrays 58 (e.g., the third transducer array 58c and the fourth transducer array 58d), the method 200 may further comprise activating the electric field generator 42 to supply the AC electrical signal having the frequency in the range from 50 kHz to 1 MHz to the second pair of transducer arrays 58, thereby generating the electric field between the second pair of transducer arrays 58 such that the second designated location (i.e., the other one of the potential probe tip locations 102) is within the electric field for a second period of time.

The second period of time may be at least a sufficient period of time for the second probe tip 90b of the second ablation probe 86b to heat to a therapeutic temperature (i.e., a temperature adequate for ablation). In some embodiments, the method 200 further comprises, subsequent to the period of time elapsing, deactivating the electric field generator 42 to cease supplying the AC electrical signal to the second pair of transducer arrays 58.

In some embodiments where the system 38a includes a first ablation probe 86a and a second probe 86b, the method 200 may further comprise: placing the second ablation probe 86b within the patient 94 such that a second probe tip 90b of the second ablation probe 86b is disposed at a second designated location (i.e., another one of the potential probe tip locations 102) within the patient 94.

In some embodiments where the system 38a includes a first pair of transducer arrays 58 (e.g., the first transducer array 58a and the second transducer array 58b) and a second pair of transducer arrays 58 (e.g., the third transducer array 58c and the fourth transducer array 58d), the method 200 may further comprise activating the electric field generator 42 to supply the AC electrical signal having the frequency in the range from 50 kHz to 1 MHz to the second pair of transducer arrays 58, thereby generating the electric field between the second pair of transducer arrays 58 such that the second designated location (i.e., the other one of the potential probe tip locations 102) is within the electric field for the second period of time.

In some embodiments, the method 200 further comprises determining a temperature at the probe tip 90 based on a resistance reading indicative of the resistance of the temperature sensor 82. In such embodiments, the method 200 may further comprise: determining whether the temperature at the probe tip 90 is above a predetermined threshold (step 216); and responsive to a determination that the temperature at the probe tip 90 is above the predetermined threshold, deactivate the electric field generator 42 to cease supplying the AC electrical signal to the one or more pair of transducer arrays 58 (step 220). The processor 66 may monitor the temperature of the probe tip 90 and actuate the electric field generator 42 to begin supplying the AC electrical signal to the one or more pair of transducer arrays 58 when the temperature of the probe tip 90 is below the predetermined threshold.

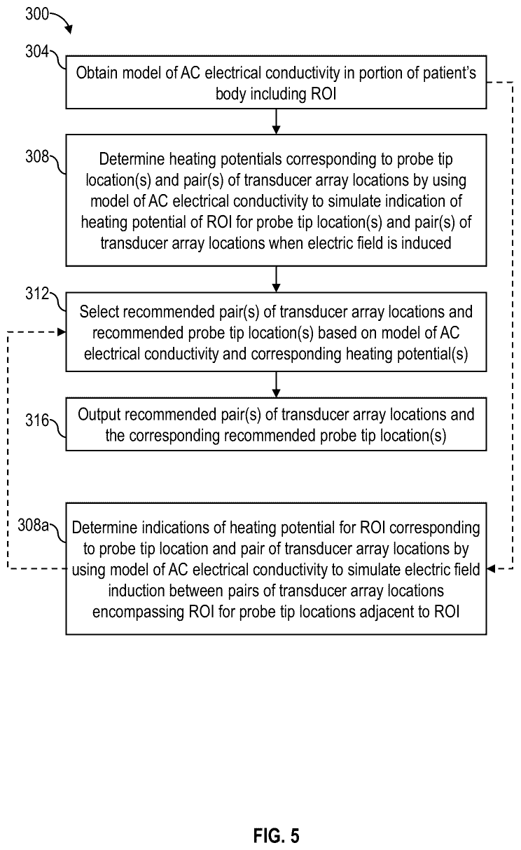

Referring now to FIG. 5, shown therein is an exemplary embodiment of a method 300 of providing recommendations for placement of the transducer arrays 58 and the ablation probe 86 in accordance with the present disclosure. As shown in FIG. 5, the method 300 generally comprises the steps of: obtaining a model of AC electrical conductivity in a portion of a body of a patient 94 including the region of interest 106 (step 304); determining a plurality of heating potentials with each heating potential corresponding to the potential probe tip location 102 and a pair of potential transducer array locations 98 by using the model of AC electrical conductivity to simulate an indication of the heating potential of the region of interest 106 for potential probe tip locations 102 within the body of the patient 94 adjacent to the region of interest 106 and particular pairs of potential transducer array locations 98 encompassing the region of interest 106 when an electric field is induced between the particular pairs of potential transducer array locations 98 (step 308); selecting one or more recommended pair of potential transducer array locations 98 and one or more corresponding recommended potential probe tip location 102 based at least in part on the model of AC electrical conductivity and one or more corresponding heating potential (step 312); and output the one or more recommended pair of potential transducer array locations 98 and the one or more corresponding recommended potential probe tip location 102 (step 316).

In some embodiments, obtaining the model of AC electrical conductivity (step 304) is further defined as obtaining a three-dimensional model of AC electrical conductivity (e.g., at the frequency that will be used for the TTFields treatment) in a portion of the body of the patient 94 including the region of interest 106 using any variety of approaches that will be apparent to persons skilled in the relevant arts. The three-dimensional model of AC electrical conductivity may comprise a plurality of voxels representing the portion of the body of the patient 94 (including the region of interest 106) and may specify a conductivity of each of the plurality of voxels.

In some embodiments, determining the plurality of heating potentials (step 308) is further defined as: selecting the plurality of pairs of potential transducer array locations 98; selecting the plurality of potential probe tip locations 102; and simulating, for each particular pair of potential transducer array locations 98 and particular corresponding potential probe tip location 102, induction of an electric field between the particular pair of potential transducer array locations 98 for the particular corresponding potential probe tip location 102 using the model of AC electrical conductivity, thereby simulating a particular indication of heating potential of the region of interest 106 for the particular pair of potential transducer array locations 98 and the particular corresponding potential probe tip location 102.

In some embodiments, selecting the one or more recommended pair of potential transducer array locations 98 and the one or more corresponding recommended potential probe tip location 102 (step 312) is further defined as: ranking simulation results for each particular pair of potential transducer array locations 98 and particular corresponding potential probe tip location 102; and selecting one or more ranked simulation results as the one or more recommended pair of potential transducer array locations 98 and the one or more corresponding recommended potential probe tip location 102. In some such embodiments, ranking the simulation results is further defined as ranking the simulation results for each particular pair of potential transducer array locations 98 and particular corresponding potential probe tip location 102 based on a particular heating potential for the particular pair of potential transducer array locations 98 and the particular corresponding potential probe tip location 102.

In some embodiments, the method 300 comprises the step of, rather than determining the plurality of heating potentials (step 308), determining a plurality of indications of heating potential for the region of interest 106, each particular indication of heating potential corresponding to a particular one of the potential probe tip locations 102 and a particular pair of potential transducer array locations 98, by using the model of AC electrical conductivity to simulate induction of an electric field between a plurality of pairs of potential transducer array locations 98 on the body of the patient 94 encompassing the region of interest 106 for a plurality of potential probe tip locations 102 in the patient's body adjacent to the region of interest 106 (step 308a).

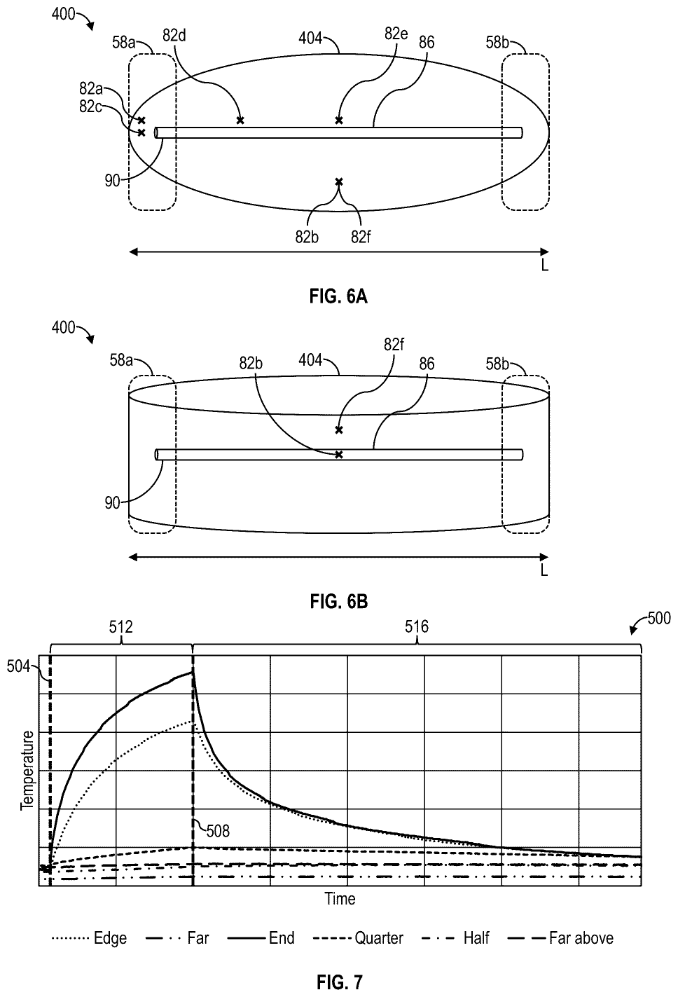

Referring now to FIGS. 6A and 6B, shown therein is an exemplary embodiment of an experimental arrangement 400 for performing validation experiments in accordance with the present disclosure. In the experimental arrangement 400, an ablation probe 86 having a probe tip 90 was disposed within a gel 404 configured to approximate human tissue. The ablation probe 86 was aligned with a longitudinal axis L, and a pair of transducer arrays—including a first transducer array 58a and a second transducer array 58b—were applied to the gel 404 on either side of the ablation probe 86 along the longitudinal axis L. In the experimental arrangement 400, the ablation probe 86 was a titanium rod having a diameter of 4.76 mm and a length of 152.4 mm. A first temperature sensor 82a, a second temperature sensor 82b, a third temperature sensor 82c, a fourth temperature sensor 82d, a fifth temperature sensor 82e, and a sixth temperature sensor 82f were disposed within the gel 404 at an edge position, a far position, an end position, a quarter position, a half position, and a far above position, respectively, relative to the ablation probe 86. An alternating current signal having a voltage of 50V was supplied to the first transducer array 58a and the second transducer array 58b to generate an electric field within the gel 404.

Referring now to FIG. 7, shown therein is a graph 500 of experimental test results determined using the experimental arrangement 400 in accordance with the present disclosure. In the graph 500, the horizontal axis represents time and the vertical axis represents temperatures as measured by the temperature sensors 82. At a first instant of time 504, the electric field generator 42 was activated and began supplying an AC electrical signal having a frequency of 200 KHz and current of 1.5 A to the transducer arrays 58, thereby generating an electric field between the transducer arrays 58 such that the gel 404—and therefore, the ablation probe 86 having the probe tip 90—was disposed within the electric field. At a second instant of time 508, the electric field generator 42 was deactivated and ceased supplying the AC electrical signal to the transducer arrays 58, thereby causing the electric field between the transducer arrays 58 to collapse. Thus, the probe tip 90 was disposed within an electric field for a first period of time 512 of approximately 23 minutes and was disposed outside of an electric field for a second period of time 516 thereafter.

As shown in FIG. 7, at the positions closest to the probe tip 90 (i.e., the first temperature sensor 82a disposed at the edge position and the third temperature sensor 82c disposed at the end position), the temperature increased sharply during the first period of time 512 to a maximum temperature of 47.80 degrees Centigrade. It is believed that the temperature adjacent to the probe tip 90 would continue increasing if the probe tip 90 were disposed within the electric field for a longer period of time, thereby reaching a therapeutic temperature (i.e., a temperature suitable for ablation). Further, at the positions along the length of the ablation probe 86 (i.e., the fourth temperature sensor 82d disposed at the quarter position and the fifth temperature sensor 82e disposed at the half position), the temperature increased slightly, indicating a low thermal conductivity of material. Finally, at the positions situated away from the ablation probe 86 (i.e., the second temperature sensor 82b disposed at the far position and the sixth temperature sensor 82f disposed at the far above position), the temperature increased minimally, which is believed to be related to heat propagation within the gel 404 and/or instability in the readings of the temperature sensors 82.

### NON-LIMITING ILLUSTRATIVE EMBODIMENTS OF THE INVENTIVE CONCEPT(S)

The following is a number list of non-limiting illustrative embodiments of the inventive concepts disclosed herein:

Illustrative embodiment 1. A method, comprising: applying one or more pair of transducer arrays to a skin surface of a patient; placing a probe within the patient such that a probe tip of the probe is disposed at a designated location within the patient, the designated location being between the one or more pair of transducer arrays, the probe tip configured to heat when disposed within an electric field; and activating an electric field generator to supply an alternating current (AC) electrical signal having a frequency in a range from 50 kHz to 1 MHz to the one or more pair of transducer arrays, thereby generating the electric field between the one or more pair of transducer arrays such that the designated location is within the electric field for a period of time.

Illustrative embodiment 2. The method of illustrative embodiment 1, wherein the probe is a first probe, the probe tip is a first probe tip, the designated location is a first designated location, and the method further comprises placing a second probe within the patient such that a second probe tip of the second probe is disposed at a second designated location within the patient.

Illustrative embodiment 3. The method of illustrative embodiment 2, wherein the period of time is at least a sufficient period of time for the probe tip to heat to a therapeutic temperature, and the method further comprises, subsequent to the period of time elapsing, deactivating the electric field generator to cease supplying the AC electrical signal to the one or more pair of transducer arrays.

Illustrative embodiment 4. The method of illustrative embodiment 3, wherein the one or more pair of transducer arrays includes a first pair of transducer arrays and a second pair of transducer arrays, the first designated location being between the first pair of transducer arrays, the second designated location being between the second pair of transducer arrays, and activating the electric field generator is further defined as activating the electric field generator to supply the al electrical signal having the frequency in the range from 50 kHz to 1 MHz to the first pair of transducer arrays, thereby generating the electric field between the first pair of transducer arrays such that the first designated location is within the electric field for the period of time.

Illustrative embodiment 5. The method of illustrative embodiment 4, wherein the period of time is a first period of time, and the method further comprises activating the electric field generator to supply the AC electrical signal having the frequency in the range from 50 kHz to 1 MHz to the second pair of transducer arrays, thereby generating the electric field between the second pair of transducer arrays such that the second designated location is within the electric field for a second period of time.

Illustrative embodiment 6. The method of illustrative embodiment 5, wherein the second period of time is at least the sufficient period of time for the probe tip to heat to the therapeutic temperature, and the method further comprises, subsequent to the second period of time elapsing, deactivating the electric field generator to cease supplying the AC electrical signal to the second pair of transducer arrays.

Illustrative embodiment 7. The method of illustrative embodiment 3, wherein applying the one or more pair of transducer arrays to the skin surface of the patient is further defined as applying the one or more pair of transducer arrays to a first position on the skin surface of the patient, the first designated location being between the one or more pair of transducer arrays in the first position, activating the electric field generator is further defined as activating the electric field generator to supply the AC electrical signal having the frequency in the range from 50 kHz to 1 MHz to the one or more pair of transducer arrays in the first position, thereby generating the electric field between the one or more pair of transducer arrays in the first position such that the first designated location is within the electric field for the period of time, and the method further comprises, subsequent to deactivating the electric field generator, moving the one or more pair of transducer arrays to a second position on the skin surface of the patient, the second designated location being between the one or more pair of transducer arrays in the second position.

Illustrative embodiment 8. The method of illustrative embodiment 7, wherein the period of time is a first period of time, and the method further comprises activating the electric field generator to supply the AC electrical signal having the frequency in the range from 50 kHz to 1 MHz to the one or more pair of transducer arrays in the second position, thereby generating the electric field between the one or more pair of transducer arrays in the second position such that the second designated location is within the electric field for a second period of time.

Illustrative embodiment 9. The method of illustrative embodiment 8, wherein the second period of time is at least the sufficient period of time for the probe tip to heat to the therapeutic temperature, and the method further comprises, subsequent to the second period of time elapsing, deactivating the electric field generator to cease supplying the AC electrical signal to the one or more pair of transducer arrays in the second position.

Illustrative embodiment 10. The method of illustrative embodiment 1, wherein the probe further comprises a thermistor adjacent to the probe tip, wherein the thermistor is a variable resistor having a resistance that varies based on temperature, and the method further comprises determining a temperature at the probe tip based on a resistance reading indicative of the resistance of the thermistor.

Illustrative embodiment 11. The method of illustrative embodiment 10, further comprising: determining whether the temperature at the probe tip is above a predetermined threshold; and responsive to a determination that the temperature at the probe tip is above the predetermined threshold, deactivate the electric field generator to cease supplying the AC electrical signal to the one or more pair of transducer arrays.

Illustrative embodiment 12. A system, comprising: an electric field generator operable to generate an alternating current (AC) electrical signal having a frequency in a range from 50 kHz to 1 MHz; one or more pair of transducer arrays configured to be electrically connected to the electric field generator and operable to generate an electric field based on the AC electrical signal; and a probe having a probe tip sized and dimensioned for insertion into a patient's body.

Illustrative embodiment 13. The system of illustrative embodiment 12, wherein the one or more pair of transducer arrays includes a first pair of transducer arrays and a second pair of transducer arrays.

Illustrative embodiment 14. The system of illustrative embodiment 12, wherein the probe further comprises a thermistor adjacent to the probe tip, wherein the thermistor is a variable resistor having a resistance that varies based on temperature.

Illustrative embodiment 15. The system of illustrative embodiment 14, further comprising a controller configured to communicate with the electric field generator and the thermistor, the controller having a processor and a non-transitory processor-readable medium storing processor-executable instructions that when executed by the processor cause the processor to: activate the electric field generator to supply the AC electrical signal to the one or more pair of transducer arrays, thereby generating an electric field between the one or more pair of transducer arrays such that the probe tip is disposed within the electric field for a period of time; and receive a resistance reading indicative of the resistance of the thermistor.

Illustrative embodiment 16. The system of illustrative embodiment 15, wherein the processor-executable instructions when executed by the processor further cause the processor to: determine whether a temperature at the probe tip is above a predetermined threshold based on the resistance reading; and responsive to a determination that the temperature at the probe tip is above the predetermined threshold, deactivate the electric field generator to cease supplying the AC electrical signal to the one or more pair of transducer arrays.

Illustrative embodiment 17. A kit, comprising: an electric field generator operable to generate an alternating current (AC) electrical signal having a frequency in a range from 50 kHz to 1 MHz; a pair of transducer arrays configured to be electrically connected to the electric field generator and operable to generate an electric field based on the AC electrical signal; and a probe having a probe tip operable to generate heat when disposed within the electric field.

Illustrative embodiment 18. The kit of illustrative embodiment 17, wherein the probe further comprises a thermistor adjacent to the probe tip, wherein the thermistor is a variable resistor having a resistance that varies based on temperature.

Illustrative embodiment 19. The kit of illustrative embodiment 18, further comprising a controller configured to communicate with the electric field generator and the thermistor, the controller having a processor and a non-transitory processor-readable medium storing processor-executable instructions that when executed by the processor cause the processor to: activate the electric field generator to supply the AC electrical signal to the one or more pair of transducer arrays, thereby generating an electric field between the pair of transducer arrays such that the probe tip is disposed within the electric field for a period of time; and receive a resistance reading indicative of the resistance of the thermistor.

Illustrative embodiment 20. The kit of illustrative embodiment 19, wherein the processor-executable instructions when executed by the processor further cause the processor to: determine whether a temperature at the probe tip is above a predetermined threshold based on the resistance reading; and responsive to a determination that the temperature at the probe tip is above the predetermined threshold, deactivate the electric field generator to cease supplying the AC electrical signal to the pair of transducer arrays.

Illustrative embodiment 21. A non-transitory processor-readable medium storing processor-executable instructions that when executed by a processor cause the processor to: obtain a model of alternating current (AC) electrical conductivity in a portion of a patient's body including a region of interest; determine a plurality of heating potentials with each heating potential corresponding to a probe tip location and a pair of transducer array locations, by using the model of AC electrical conductivity to simulate an indication of the heating potential of the region of interest for a plurality of probe tip locations within the patient's body adjacent to the region of interest and particular pairs of transducer array locations of a plurality of pairs of transducer array locations encompassing the region of interest when an electric field is induced between the particular pairs of transducer array locations; select one or more recommended pair of transducer array locations and one or more corresponding recommended probe tip location based at least in part on the model of AC electrical conductivity and one or more corresponding heating potential; and output the one or more recommended pair of transducer array locations and the one or more corresponding recommended probe tip location.

Illustrative embodiment 22. The non-transitory processor-readable medium of illustrative embodiment 21, wherein determining the plurality of heating potentials is further defined as: selecting the plurality of pairs of transducer array locations; selecting the plurality of probe tip locations; and simulating, for each particular pair of transducer array locations and particular corresponding probe tip location, induction of an electric field between the particular pair of transducer array locations for the particular corresponding probe tip location using the model of AC electrical conductivity, thereby simulating a particular indication of heating potential of the region of interest for the particular pair of transducer array locations and the particular corresponding probe tip location.

Illustrative embodiment 23. The non-transitory processor-readable medium of illustrative embodiment 22, wherein selecting the one or more recommended pair of transducer array locations and the one or more corresponding recommended probe tip location is further defined as: ranking simulation results for each particular pair of transducer array locations and particular corresponding probe tip location; and selecting one or more ranked simulation results as the one or more recommended pair of transducer array locations and the one or more corresponding recommended probe tip location.

Illustrative embodiment 24. The non-transitory processor-readable medium of illustrative embodiment 22, wherein ranking the simulation results is further defined as ranking the simulation results for each particular pair of transducer array locations and particular corresponding probe tip location based on a particular heating potential for the particular pair of transducer array locations and the particular corresponding probe tip location.

Illustrative embodiment 25. The non-transitory processor-readable medium of illustrative embodiment 21, wherein determining the plurality of heating potentials is defined as determining a plurality of indications of heating potential for the region of interest, each particular indication of heating potential corresponding to a particular probe tip location and a particular pair of transducer array locations, by using the model of AC electrical conductivity to simulate induction of an electric field between a plurality of pairs of transducer array locations on the patient's body encompassing the region of interest for a plurality of probe tip locations in the patient's body adjacent to the region of interest.

### CONCLUSION

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A method, comprising:
applying one or more pair of transducer arrays to a skin surface of a patient;
placing a probe within the patient such that a probe tip of the probe is disposed at a designated location within the patient, the designated location being between the one or more pair of transducer arrays, the probe tip configured to heat when disposed within an electric field; and
activating an electric field generator to supply an alternating current (AC) electrical signal having a frequency in a range from 50 kHz to 1 MHz to the one or more pair of transducer arrays, thereby generating the electric field between the one or more pair of transducer arrays such that the designated location is within the electric field for a period of time.

2. The method of claim 1, wherein the probe is a first probe, the probe tip is a first probe tip, the designated location is a first designated location, and the method further comprises placing a second probe within the patient such that a second probe tip of the second probe is disposed at a second designated location within the patient.

3. The method of claim 2, wherein the period of time is at least a sufficient period of time for the probe tip to heat to a therapeutic temperature, and the method further comprises, subsequent to the period of time elapsing, deactivating the electric field generator to cease supplying the AC electrical signal to the one or more pair of transducer arrays.

4. The method of claim 3, wherein the one or more pair of transducer arrays includes a first pair of transducer arrays and a second pair of transducer arrays, the first designated location being between the first pair of transducer arrays, the second designated location being between the second pair of transducer arrays, and activating the electric field generator is further defined as activating the electric field generator to supply the AC electrical signal having the frequency in the range from 50 kHz to 1 MHz to the first pair of transducer arrays, thereby generating the electric field between the first pair of transducer arrays such that the first designated location is within the electric field for the period of time.

5. The method of claim 4, wherein the period of time is a first period of time, and the method further comprises activating the electric field generator to supply the AC electrical signal having the frequency in the range from 50 kHz to 1 MHz to the second pair of transducer arrays, thereby generating the electric field between the second pair of transducer arrays such that the second designated location is within the electric field for a second period of time.

6. The method of claim 5, wherein the second period of time is at least the sufficient period of time for the probe tip to heat to the therapeutic temperature, and the method further comprises, subsequent to the second period of time elapsing, deactivating the electric field generator to cease supplying the AC electrical signal to the second pair of transducer arrays.

7. The method of claim 3, wherein applying the one or more pair of transducer arrays to the skin surface of the patient is further defined as applying the one or more pair of transducer arrays to a first position on the skin surface of the patient, the first designated location being between the one or more pair of transducer arrays in the first position, activating the electric field generator is further defined as activating the electric field generator to supply the AC electrical signal having the frequency in the range from 50 kHz to 1 MHz to the one or more pair of transducer arrays in the first position, thereby generating the electric field between the one or more pair of transducer arrays in the first position such that the first designated location is within the electric field for the period of time, and the method further comprises, subsequent to deactivating the electric field generator, moving the one or more pair of transducer arrays to a second position on the skin surface of the patient, the second designated location being between the one or more pair of transducer arrays in the second position.

8. The method of claim 7, wherein the period of time is a first period of time, and the method further comprises activating the electric field generator to supply the AC electrical signal having the frequency in the range from 50 kHz to 1 MHz to the one or more pair of transducer arrays in the second position, thereby generating the electric field between the one or more pair of transducer arrays in the second position such that the second designated location is within the electric field for a second period of time.

9. The method of claim 8, wherein the second period of time is at least the sufficient period of time for the probe tip to heat to the therapeutic temperature, and the method further comprises, subsequent to the second period of time elapsing, deactivating the electric field generator to cease supplying the AC electrical signal to the one or more pair of transducer arrays in the second position.

10. The method of claim 1, wherein the probe further comprises a thermistor adjacent to the probe tip, wherein the thermistor is a variable resistor having a resistance that varies based on temperature, and the method further comprises determining a temperature at the probe tip based on a resistance reading indicative of the resistance of the thermistor.

11. The method of claim 10, further comprising:
determining whether the temperature at the probe tip is above a predetermined threshold; and
responsive to a determination that the temperature at the probe tip is above the predetermined threshold, deactivate the electric field generator to cease supplying the AC electrical signal to the one or more pair of transducer arrays.

12. A system, comprising:
an electric field generator operable to generate an alternating current (AC) electrical signal having a frequency in a range from 50 kHz to 1 MHz;
one or more pair of transducer arrays configured to be electrically connected to the electric field generator and operable to generate an electric field based on the AC electrical signal; and
a probe having a probe tip sized and dimensioned for insertion into a patient's body.

13. The system of claim 12, wherein the one or more pair of transducer arrays includes a first pair of transducer arrays and a second pair of transducer arrays.

14. The system of claim 12, wherein the probe further comprises a thermistor adjacent to the probe tip, wherein the thermistor is a variable resistor having a resistance that varies based on temperature.

15. The system of claim 14, further comprising a controller configured to communicate with the electric field generator and the thermistor, the controller having a processor and a non-transitory processor-readable medium storing processor-executable instructions that when executed by the processor cause the processor to:
activate the electric field generator to supply the AC electrical signal to the one or more pair of transducer arrays, thereby generating an electric field between the one or more pair of transducer arrays such that the probe tip is disposed within the electric field for a period of time; and
receive a resistance reading indicative of the resistance of the thermistor.

16. The system of claim 15, wherein the processor-executable instructions when executed by the processor further cause the processor to:
determine whether a temperature at the probe tip is above a predetermined threshold based on the resistance reading; and
responsive to a determination that the temperature at the probe tip is above the predetermined threshold, deactivate the electric field generator to cease supplying the AC electrical signal to the one or more pair of transducer arrays.

17. A kit, comprising:
an electric field generator operable to generate an alternating current (AC) electrical signal having a frequency in a range from 50 kHz to 1 MHz;
a pair of transducer arrays configured to be electrically connected to the electric field generator and operable to generate an electric field based on the AC electrical signal; and
a probe having a probe tip operable to generate heat when disposed within the electric field.

18. The kit of claim 17, wherein the probe further comprises a thermistor adjacent to the probe tip, wherein the thermistor is a variable resistor having a resistance that varies based on temperature.

19. The kit of claim 18, further comprising a controller configured to communicate with the electric field generator and the thermistor, the controller having a processor and a non-transitory processor-readable medium storing processor-executable instructions that when executed by the processor cause the processor to:
activate the electric field generator to supply the AC electrical signal to the one or more pair of transducer arrays, thereby generating an electric field between the pair of transducer arrays such that the probe tip is disposed within the electric field for a period of time; and
receive a resistance reading indicative of the resistance of the thermistor.

20. The kit of claim 19, wherein the processor-executable instructions when executed by the processor further cause the processor to:
determine whether a temperature at the probe tip is above a predetermined threshold based on the resistance reading; and
responsive to a determination that the temperature at the probe tip is above the predetermined threshold, deactivate the electric field generator to cease supplying the AC electrical signal to the pair of transducer arrays.
